(19) United States
(12) Patent Application Publication
(10) Pub. No.: US 2001/0046668 A1
(43) Pub. Date: Nov. 29, 2001

(54) Fluorescence polarization method for determining protease activity

(75) Inventors: Leanna M. Levine, Olivette, MO (US); Mihaly V. Toth, St. Louis, MO (US)

Correspondence Address: Pharmacia Corporation, Corporate Patent Department, 800 North Lindbergh Blvd., Mail Zone O4E, St. Louis, MO, 63167, US

(73) Assignee: G.D. Searle & Co., Chicago, IL (US)

(21) Appl. No.: 09/880,654

(22) Filed: Jun. 13, 2001

Related U.S. Application Data
(63) Continuation of application No. 09/246,787, filed on Feb. 8, 1999, now abandoned.
Application No. 09/246,787 is a continuation of application No. 08/867,258, filed on Jun. 2, 1997, now abandoned.
Application No. 08/867,258 is a continuation of application No. 08/327,170, filed on Oct. 21, 1994, now abandoned.

(51) Int. Cl. C12Q 1/70; G01N 33/53; C07K 5/00; A61K 38/04

(52) U.S. Cl. 435/5; 435/7.1; 435/23; 436/518; 435/9; 530/326; 530/328

(57) ABSTRACT

A fluorescence polarization method of determining protease activity is described.

### FIELD OF THE INVENTION

This invention relates to a method for determining the activity of proteases. More particularly, the invention relates to a fluorescence polarization assay for viral proteases.

### BACKGROUND OF THE INVENTION

There is a great need for new therapies for the treatment of viral diseases. Whereas there has been great progress in developing a variety of therapies for the treatment of bacterial infections, there are few viable therapies for the treatment of viruses in general, and herpesvirus and HIV in particular.

It is known that some viruses express their genetic content by directing the synthesis of a number of proteins encoded by the virus DNA in the host cell. One of the important herpesvirus encoded proteins is made as a precursor consisting of an amino terminal-located protease and carboxyl terminal-located assembly protein. This precursor is proteolytically processed in an autocatalytic manner at a specific amino acid sequence known as the “release” site yielding separate protease and assembly protein. The assembly protein is cleaved further by the protease at another specific amino acid sequence known as the “maturation” cleavage site. Recently, a virus-specific serine protease which has a role in herpesvirus replication has been described. A. R. Welch et al (Proc. Natl. Acad. Sci. USA, 88, 10792 (1991)) describe the related protease (also known as assemblin) and assembly protein encoded by U_{L}80 of CMV. An approach currently being investigated for potential use in the treatment of herpesvirus infections is the development of inhibitors of herpesvirus proteases.

Similarly, RNA viruses, such as retroviruses, encode proteins which are processed by expressed viral proteases. During the replication cycle of retroviruses, gag and gag-pol gene transcription products are translated as proteins. These proteins are subsequently processed by a virally encoded protease to yield viral enzymes and structural proteins of the viral core. Most commonly, the gag precursor proteins are processed into the core proteins and the pol precursor proteins are processed into the viral enzymes, e.g., reverse transcriptase and retroviral protease. It has been shown that correct processing of the precursor proteins by the retroviral protease is necessary for assembly of infectious virons. Thus, attempts have been made to inhibit viral replication by inhibiting the action of retroviral proteases.

In order to facilitate the rapid identification of virus protease inhibitors, an assay which allows for high throughput and linearity is desirable. In addition, a viral assay could be used to diagnose patients having viral infections.

Initial assays used in the characterization of herpesvirus proteases have been based on electrophoretic separation of products. See EP 514,830. Such method is impractical for screening large numbers of enzymatic inhibitors. An assay which allows for quantitative kinetic characterization of the interaction of the inhibitors with herpesvirus proteases is more preferred.

The use of fluorogenic substrates for the determination of protease activities has been described. U.S. Pat. No. 5,011,910, to G. Marshall and M. Toth, describes the use of fluorogenic substrates for the determination of HIV proteases. E. Matayoshi et al describe the use of an EDANS/DABCYL-containing substrate for assaying HIV protease (Science, 247, 954 (1990)). L. Maggiora et al describe a solid-phase peptide synthesis method of preparing EDANS/DABCYL-containing substrates (J. Med. Chem., 35, 3727 (1992)). However, fluorescence based assays, especially for screening natural products libraries, have many disadvantages associated with them, especially interference from high fluorescence background.

Fluorescence polarization is a detection method that ratios the intensities of vertically versus horizontally polarized fluorescence from a sample that has been illuminated with plane polarized light. Fluorescence polarization techniques have been described for the study of enzyme activity. A. Ping and J. Herron describe a competitive fluorescent polarization immunoassay wherein a fluorescent peptide substrate is displaced from an antibody by a natural substrate of interest (Anal. Chem., 65, 3372-3377 (1993)). U.S. Pat. No. 5,070,025, to Klein et al, describes a fluorescence polarization immunoassay. U.S. Pat. No. 4,640,893, to Mangel et al, describes rhodamine-peptide derivatives as fluorogenic protease substrates. H. Maeda describes the use of fluorescence polarization in the study of proteolytic enzyme cleavage of protein substrates (Anal. Biochem., 92, 222-227 (1979)). H. Maeda describes the use of fluorescence polarization in the study of lysozyme cleavage of an isolated peptidoglycan natural product(J. Biochem., 88, 1185-1191 (1980)).

Protease activity measurements by fluorescence polarization using peptide substrates containing biotin and fluorescein radicals is described by R. Bolger and W. Checovich (Biotechniques, 17, 585-89 (1994)).

### BRIEF DESCRIPTION OF THE FIGURES

FIG. 1 is a graphical representation which shows the effect of substrate cleavage on fluorescence polarization. The biotin-γ-Abu-Gly-Val-Val-Asn-Ala-Arg-Ser-Leu-Lys(DTAF)-NH₂ substrate is shown bound to avidin. Avidin-bound uncleaved peptide has a high polarization value whereas the cleaved peptide has a lower value.

FIG. 2 is a graphical representation which shows the reduction of fluorescence polarization due to the hydrolysis of biotin-γ-Abu-Gly-Val-Val-Asn-Ala-Arg-Ser-Leu-Lys(DTAF)-NH₂ [SEQ ID NO:3] substrate by the HCMV protease encoded by U_{L}80 (also known as assemblin). Fluorescence polarization magnitude (mP) is plotted versus time (minutes).

FIG. 3 is a graphical representation which shows the linear change in fluorescence polarization (ΔP) from the HCMV assemblin protease hydrolysis of biotin-γ-Abu-Gly-Val-Val-Asn-Ala-Arg-Ser-Leu-Lys(DTAF)-NH₂ [SEQ ID NO:3] substrate. The change in polarization in mP is plotted versus time (minutes).

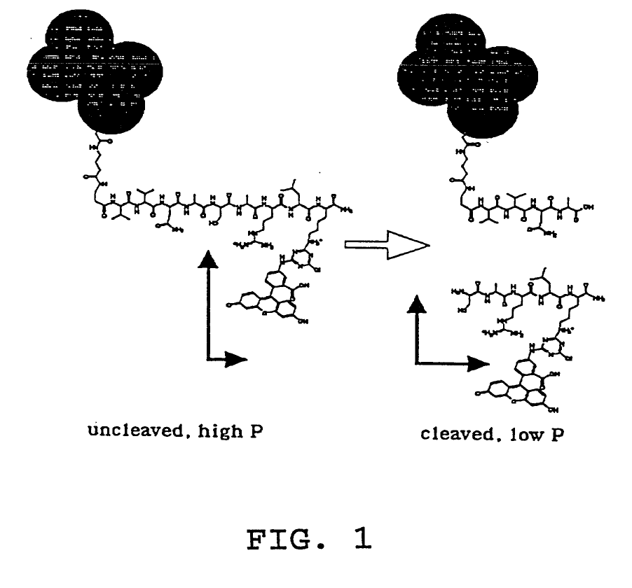

### SUMMARY OF THE INVENTION

A fluorescence polarization method is disclosed for determining the activity of a protease. The method comprises incubating a mixture of a protease of interest and a protease-selective substrate so that the protease may cleave the substrate. The substrate is capable of being bound to an anchor, and the substrate also includes a fluorescent radical. After the incubation period, the substrate is attached to the anchor, if not previously attached. The amount of cleaved substrate is determined by monitoring the change in the total fluorescence polarization of the mixture.

The enzyme concentration used for this assay is 5 times lower than that achievable conveniently with other assays. This assay has the advantage of being a solution phase determination of enzyme activity and requires no further manipulations other than addition of reagents at the appropriate times. It is appropriate for adaptation in a high throughput automated or semi-automated assay, and especially for a natural products screen since the polarization signal is derived from the ratio of fluorescence intensities and is less sensitive to contributions from background fluorescence. One can thus determine protease activity in the presence of high fluorescence background.

### DETAILED DESCRIPTION OF THE INVENTION

In accordance with the present invention, a method for determining the activity of a protease is described, the method comprising

a) incubating a mixture of said protease and a substrate capable of being bound to an anchor, said substrate having a fluorescent radical attached thereto;

b) binding the substrate to an anchor;

c) measure the fluorescence polarization of the mixture.

Preferably, the substrate is selected from compounds of Formula I

Z—(W)ₘ—X—(V)ₙ—Y (I)

wherein X is an amino acid sequence sufficient for substrate recognition by a protease; wherein V and W are independently selected from aminoalkylcarboxylic acids; wherein m and n are numbers independently selected from 0 and 1; and wherein one of Y and Z is a fluorescent radical and the other is a binding radical.

The length of the peptide is limited only by the requirements of peptide activity with the enzyme. Any length peptide may be used that shows both enzyme activity and a change of polarization upon hydrolysis that is measurable. More preferably, X is a peptide containing six to sixteen amino acids, inclusive; wherein V and W are independently selected from glycine, 4-aminobutyric acid, 5-aminopentanoic acid, 6-aminocaproic acid and 7-aminoheptanoic acid.

The anchor is selected from a radical-selective protein, a solid support, and an antibody. Such anchors can include proteins such as avidin and streptavidin, polymeric supports, substrate-related antibodies such as anti-digoxigenin, glass beads, paper, membranes, gels, metals, and the like. The substrate may be attached to the anchor prior to mixing with the protease if the anchor will not interfere with cleavage of the substrate, or can be attached after protease contact. The specific peptide substrate can be attached to another protein or polymer through standard linking chemistries, such as glutaraldehyde, carbodiimides, and the like (see van Regenmortel et al, Synthetic Polypeptides as Antigens, 1988) and avoid the use of a binding pair. The peptide substrate also can be covalently attached to or non-specifically adsorbed to a bead or gold microparticle. Preferably, the anchors are radical selective proteins, such as avidin or streptavidin.

Preferably, the binding radical is selected from radicals which selectively bind to proteins or are able to be connected to the above anchors. More preferably, the binding radical is selected from digoxigenin and biotin, and even more preferably, is biotin.

Any fluorescent radical can be used which has a measurable fluorescence polarization somewhere in the excitation spectrum. Preferably, the fluorescent radical is selected from cascade blue, Texas red, acidine orange, fluorescein, rhodamine, coumarin, eosin, pyrene, quinoline, DANSYL, dinitrophenyl, benzimidazole, DABCYL, EDANS, BODIPY and derivatives thereof. More preferably, the fluorescent radical belongs to the family of fluorescein dyes. Even more preferably, the fluorescent radical is DTAF.

The method of the current invention is appropriate for the evaluation of proteases from viruses. The method is particularly appropriate wherein the proteases from HIV or herpes. Such herpes viruses include HCMV, MCMV, HSV-1 and HSV-2, among others.

The assay can be performed in a physiological buffer. Preferably, the buffer consists of 10 mM phosphate buffer. The specific buffer conditions may change with the protease involved, but for HIV protease as well as a HCMV protease (also known as assemblin) encoded by U_{L}80, glycerol is preferred. Preferably, the pH of the buffer is adjusted before addition of any glycerol.

Protease is stored as a 10 μM stock solution in 50/50 (V/V) glycerol/water, 50 μL per vial, and held at −20° C. A positive displacement pipette is used to make the 50 μL aliquots. This stock is diluted with assay buffer to 20-30 μM. About 100 μL of this solution is used in the assay.

A 150 μM substrate stock solution is prepared in assay buffer and stored at 4° C. in the dark. A 20 μM dilute assay stock is prepared by dilution of the 150 μM storage stock.

A 5 mg/mL avidin stock solution is made by dissolving avidin (Molecular Probes) in assay buffer and stored at 4° C.

Microtiter plates (Black MicroFluor, Dynatech) are pre-blocked with 1.0% BSA in PBS, pH 7.4, and stored at 4° C. The plates are rinsed and dried before use.

Preferably, to wells of rinsed, dried, and pre-blocked roundbottom microtiter plates, a known amount of protease in buffer is added. No precautions are taken to stabilize the temperature. Substrate is added, the resulting mixture is mixed 5 times, such as by pipette, and incubated for about 1.5 hours at room temperature. Avidin is added, the resulting mixture is mixed 5 times, and the polarization is measured on a fluorescence polarimeter (FPM2, Jolley Consulting and Research). Runs are made with duplicate wells.

Mixing at least five times is important to insure thorough mixing of reagents in a buffer containing 20% glycerol. It is recommend to transfer a minimum of 25 μL of any reagent in buffer containing glycerol to insure repeatability of volumetric additions.

Raw intensity data is transferred from the polarimeter via RS232 serial data line to a Macintosh using an ELISAREAD Excel® spreadsheet customized for this application.

The polarization measured is directly proportional to the concentrations of the cleaved and uncleaved peptide. The polarization (P) can be calculated with Equation 1:

P=(Iᵥ−G·Iₕ)/(Iᵥ+G·Iₕ) (1)

wherein Iᵥ is the vertical polarization emission intensity, Iₕ is the horizontal polarization emission intensity, and G is the instrument factor that corrects for polarization introduced by the instrument optics and the light source.

The total fluorescence (I_{Tot}) is also readily determined by calculation as shown in Equation (2). A direct measure of product formed can be determined from the anisotropy (A), calculated as shown below Equation (3), and a direct measure of product formed or substrate consumed can be determined by Equation (4):

I_{Tot}=(Iᵥ+G·2Iₕ) (2)

A=(Iᵥ−G·Iₕ)/(Iᵥ+G·2Iₕ) (3)

[Product]=[Substrate]₀(1−[(Aₜ−Aₘᵢₙ)/(Aₘₐₓ−Aₘᵢₙ)])

where

Aₜ=anistropy at time=t;

Aₘᵢₙ=anistropy for the totally clipped fluorescent peptide fragment; and

Aₘₐₓ=anistropy for the bound, unclipped, peptide bound to avidin.

A significant advantage of this assay is that it has the well-known sensitivity of a traditional fluorescence assay without the sensitivity to fluorescence quenchers sometimes present, such as in natural product extracts. Since polarization measured is the ratio of the difference divided by the sum of the vertical (Iᵥ) and horizontal (Iₕ) polarization emission intensities, the total fluorescence intensity from the sample is not needed to accurately determine the polarization. The presence of quenchers and artifacts in the polarization values can be easily spotted by comparing the total fluorescence with the polarization values of the sample and the controls.

Also described is a method for identifying a compound which inhibits a protease, the method comprising a) incubating a mixture of said protease, the compound, and a substrate having both a fluorescent radical and a radical capable of binding to an anchor; b) binding the substrate to the anchor; c) measure the polarization of the fluorescent light emitted from the mixture; and d) calculating the amount of protease inhibition.

In a routine assay, inhibitor sample (about 10 μM) dissolved in a suitable solvent such as DMSO, is added in duplicate wells to the rinsed and dried, pre-blocked microtiter plate. A known amount of protease in buffer is added and the solution is mixed 5 times. Protease and inhibitor sample are incubated for about 30 minutes at room temperature. No precautions are taken to stabilize the temperature. A known amount of substrate is added, the resulting mixture is mixed 5 times, and incubated for about 1.5 hours at room temperature. Avidin (35 μL of 5 mg/mL) is added, the resulting mixture is mixed 5 times, and the fluorescence polarization is measured.

Controls for this assay include the unclipped peptide in assay buffer to produce the minimum polarization value (Pₘᵢₙ), the peptide plus avidin in buffer (Pₘₐₓ), the peptide plus enzyme in buffer, incubated for 1.5 hours and quenched with avidin (according to assay protocol) (P_{control}). The % control activity of an unknown inhibitor can be calculated as:

[(Pₘₐₓ−P_{control})/P_{control}]×100. (5)

To determine IC₅₀ of an inhibitor, the % control activity is plotted as a function of the inhibitor concentration. the IC₅₀ is either read graphically or determined by a standard curve-fitting routine.

For the HIV substrate and the HCMV protease encoded by U_{L}80 (also known as assemblin) substrate, the addition of avidin also quenches the enzyme hydrolysis. The assay is thus a solution phase end point determination of substrate cleavage. However, a continuous assay could be constructed where the peptide length is long enough or of the right spatial orientation to allow hydrolysis in the presence of the anchor.

An amino acid sequence sufficient for substrate recognition by a herpesvirus protease includes “maturation” cleavage site sequences and “release” cleavage site sequences of herpesvirus protease substrates. These include “maturation” and “release” cleavage site sequences for HCMV, HSV-1, HSV-2, VZV, HHV-6, HHV-7 and EBV proteases. The preferred novel substrates are based on a HCMV “maturation” cleavage site sequence (Val-Ala-Glu-Arg-Ala-Gln-Ala-Gly-Val-Val-Asn-Ala*Ser-Cys-Arg-Leu-Ala-Thr-Ala [SEQ ID NO:1], where “*” denotes the cleavage site) at the C-terminus of the capsid assembly protein. The gag and pol polyproteins have several cleavage sites for an HIV protease. (Abdel-Meguid, Medicinal Res. Rev., 13, 731-778 (1993)). The preferred HIV substrate sequence is Ser-Gln-Asn-Tyr*Pro-Ile-Val-Gln [SEQ ID NO:2], where “*” denotes the cleavage site. Peptides of various lengths encompassing these sequences, or homologs thereof, provide amino acid sequences sufficient for substrate recognition by a herpesvirus protease.

Other possible variations on this assay to increase its sensitivity by increasing the dynamic range may take advantage of the solution tumbling properties of molecules. Lowering the temperature could in some cases lower the tumbling of a higher molecular weight species more than a lower molecular weight species and increase the dynamic range of the polarization assay. Polarization theory also predicts that the maximum polarization possible will be a function of the relationship between the excitation and emission dipoles of the fluorophore. Thus a more sensitive assay could be envisioned by adjusting the excitation wavelength to achieve maximum polarization in the emission or by choice of fluorophore.

Although a hydrolysis incubation period of 30 to 90 minutes may be sufficient, the maximum length of incubation of enzyme with substrate is limited only by the stability of the enzyme. Assay lengths of several hours or overnight are possible for measuring the activity of a very low activity enzymes.

Where the term “fluorescent radical” is used, it embraces a fluorescence emitting radical, such as fluorescein, anthracene, aminobenzoyl, indole, and aminoethylnaphthyl radicals, and the like, which can be modified and attached to the amino acid sequence. Such radicals include cascade blue, Texas red, acidine orange, fluorescein and derivatives thereof such as 5-([4,6-dichlorotriazin-2-yl]amino) fluorescein (DTAF), rhodamine and derivatives thereof, coumarin and derivatives thereof, eosin and derivatives thereof, pyrene and derivatives thereof, quinoline and derivatives thereof, dinitrophenyl and derivatives thereof, benzimidazole and derivatives thereof, DABCYL and derivatives thereof, BODIPY and derivatives thereof, 5-[(2-aminoethyl)amino] naphthalene-1-sulfonic acid (EDANS), 2-aminobenzoic acid (Abz) and derivatives thereof, e.g. N-methyl-Abz, 4-chloro-Abz, 5-chloro-Abz, 6-chloro-Abz, 3,5-dibromo-Abz, 5-dimethylamino-naphthalene-1-sulfonyl (DANSYL) and derivatives thereof, nicotinic acid and derivatives thereof, such as 6-aminonicotinic acid, 2-aminonicotinic acid, 2-chloronicotinic acid, and niflumic acid, 4-guanidino-benzoic acid and derivatives of 4-guanidino-benzoic acid; and the like. The term “anchor” embraces materials having molecular weights sufficient to significantly different polarization as compared to the cleaved substrate portion. For a cleaved portion having a molecular weight of about 1000, an anchor having a molecular weight of about 5000 may be sufficient to generate a measurable difference in polarization. Preferably, anchors include proteins, polymeric supports, substrate-related antibodies, glass beads, membranes, gels, metals, and the like. The term “binding radical” embraces radicals which selectively bind to proteins or are able to be connected to the anchors, as defined above. Preferably, the binding radical is selected from digoxigenin and biotin. The term “aminoalkylcarboxylic acids” embraces radicals which can be included between either the fluorescing donor radical or the acceptor radical, and the peptide sequences. Such radicals act as spacers and reduce the possibility of the fluorescing donor radical or the acceptor radical, having a steric or other negative effect on the binding of the substrate and the enzyme. Such aminoalkylcarboxylic acids embrace linear or branched radicals having one to about twenty carbon atoms or, preferably, one to about twelve carbon atoms. More preferred aminoalkylcarboxylic acids radicals are “lower aminoalkylcarboxylic acids” radicals having one to about ten carbon atoms. Most preferred are lower aminoalkylcarboxylic acids radicals having one to about seven carbon atoms. Examples of such radicals include glycine, 4-aminobutyric acid, 5-aminopentanoic acid, 6-aminocaproic acid, 7-aminoheptanoic acid, and the like.

It will be appreciated that various modifications can be made to the aforesaid preferred fluorescent substrates to provide substantially similar useful results in the fluorescent polarization assay for virus proteases.

While the specification concludes with claims particularly pointing out and distinctly claiming the subject matter regarded as forming the present invention, it is believed that the invention will be better understood from the following detailed description of preferred embodiments of the invention taken in conjunction with the appended figures.

### GENERAL SYNTHETIC PROCEDURES

The preferred novel fluorogenic substrates of this invention and their analogs can be made by known solution and solid phase peptide synthesis methods but modified to incorporate the binding radical, e.g. biotin at the N-terminal position, the fluorescent radical, e.g. DTAF at the C-terminal portion, such as through a lysine side chain or through spacer radicals located between the peptide and the binding radical or the fluorescent radical. The preferred peptide synthesis method follows conventional Merrifield solid-phase procedure [J. Amer. Chem. Soc., 85, 2149 (1963); Science, 150, 178 (1965) modified by the procedure of Tam et al., J. Amer. Chem. Soc., 105, 6442 (1983)].

In order to illustrate specific preferred embodiments of the invention in greater detail, the following exemplary laboratory preparative work was carried out. It should be understood that the invention is not limited to these specific examples.

Solid phase synthesis of assemblin substrates are prepared by conventional solid phase peptide synthesis using methylbenzhydrylamine (MBHA) resin. For each synthesis, 1 gram of resin was used (0.7 mmole) . The following synthetic protocol is an example of that can be used for incorporation of the Boc-amino acids.

Deprotection:
50% trifluoroacetic acid/CH₂Cl₂ 5 minutes/25 minutes

| | |
|---|---|
| CH₂Cl₂ | 2 × 1 minutes |
| Isopropanol | 2 × 1 minutes |
| CH₂Cl₂ | 2 × 1 minutes |

Neutralization:
10% diisopropylethylamine/CH₂Cl₂ 3 minutes/5 minutes

| | |
|---|---|
| CH₂Cl₂ | 2 × 1 minutes |
| DMF | 2 × 1 minutes |

Amino acids are coupled to the resin, or the growing peptide chain on the resin, by adding 4-equivalents of butyloxycarbonyl (Boc) protected amino acid and 4 equivalents of dicyclohexylcarbodiimide (DCC) in the presence of 4 equivalents of hydroxybenzotriazole (HOBT) in dimethylformamide (DMF) for 2 hours. Biotin (Sigma) is manually coupled using benzotriazolyl-N-oxy-tris (dimethylamino)-phosphonium hexafluorophosphate (BOP) and diisopropylethylamine (DIPEA). Completed peptides are cleaved by the hydrofluoric acid (HF)/anisole 9:1 procedure of Tam et al., J. Amer. Chem. Soc., 105, 6442 (1983). Crude Biotin-peptides are dissolved in 20% acetic acid and lyophilized. Crude peptides are purified by reverse-phase HPLC on a C₁₈ semipreparative column using a 0.1% trifluoroacetic acid (TFA) and acetonitrile gradient. The 5-([4,6-dichlorotriazin-2-yl]amino)fluorescein (DTAF) radical can be coupled to the free amine of a lysine radical incorporated in the biotin-peptide amide through loss of a chloro atom by adding DTAF in DMF in the presence of diisopropylethylamine (DIPEA). The mixture was filtered and diluted with 5 mL 50% acetic acid and 20 mL water. Crude biotin/DTAF-peptides are purified by reverse-phase HPLC on a C₁₈ semipreparative column using a 0.1% TFA and acetonitrile gradient. Their identity can be confirmed by high-resolution mass spectrometry.

### EXAMPLE 1

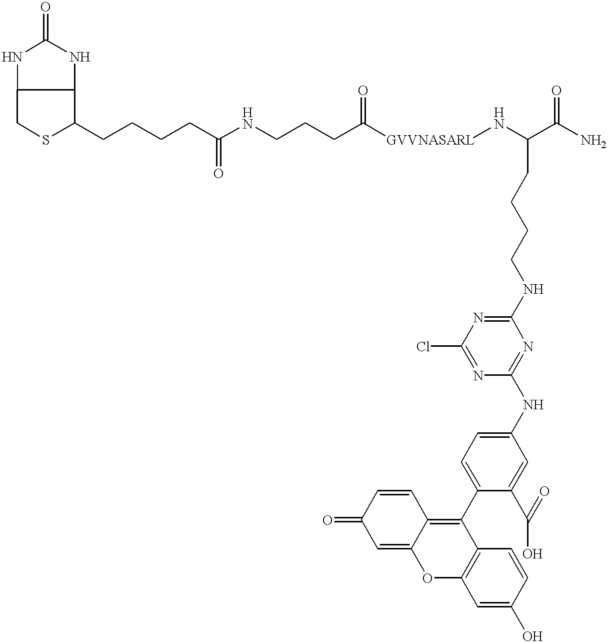

### Biotin-γ-Abu-Gly-Val-Val-Asn-Ala-Ser-Ala-Arg-Leu-Lys(DTAF)-NH₂ [SEQ ID NO:3]

The assemblin substrate peptide core was synthesized on an Applied Biosystems peptide synthesizer (Model 430A) using a standard synthesis protocol, starting with 0.72 mmol MBHA resin (1 g). Biotin (Sigma) was manually coupled using BOP and DIPEA. The fully protected Biotin-γ-Abu-peptide resin was cleaved and deprotected with treatment of HF/anisole (9:1) at 0° C. for 1 hour. Crude Biotin-γ-Abu-peptide was purified on HPLC using acetonitrile/water (0.1% TFA) gradient (20-50% acetonitrile in 30 minutes). Purified Biotin-peptide amide in 5 mL DMF (52 mg, 40 μmol) was coupled with 24.7 mg (50 μmol) DTAF in the presence of 17.5 μL (100 μmol) DIPEA for 12 hour. The reaction mixture was filtered and diluted with 5 mL 50% acetic acid and 20 mL water. This solution was purified on HPLC using acetonitrile/water (0.1% TFA) gradient (20-50% acetonitrile in 30 minutes). The identity of biotin-γ-Abu-Gly-Val-Val-Asn-Ala-Ser-Ala-Arg-Leu-Lys(DTAF)-NH₂ [SEQ ID NO:3] was confirmed by FAB mass spectrometry: (M+H)=1781.5

### EXAMPLE 2

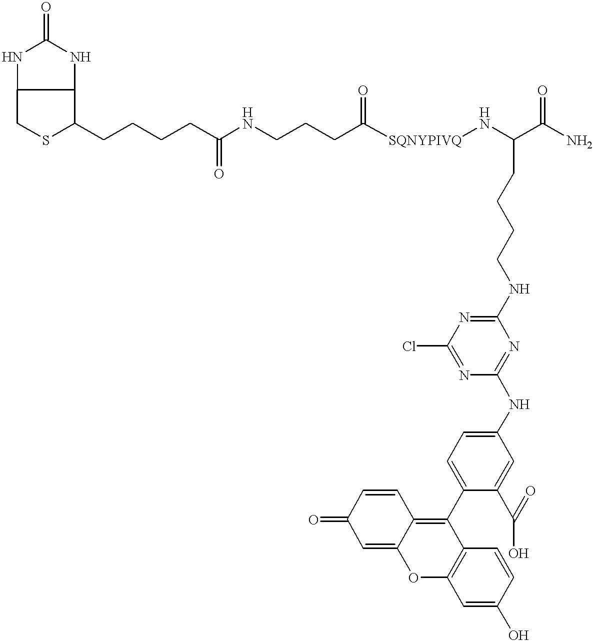

### Biotin-γ-Abu-Ser-Gln-Asn-Tyr-Pro-Ile-Val-Gln-Lys (DTAF)-NH₂ [SEQ ID NO:4]

The HIV protease peptide core was synthesized on an Applied Biosystems peptide synthesizer (Model 430A) using a standard synthesis protocol, starting with 1 g of 0.72 mmol MBHA resin. Biotin was coupled manually using BOP and DIPEA. The fully protected Biotin-peptide resin was cleaved and deprotected with treatment of HF/anisole (9:1) at 0° C. for 1 hour. Crude Biotin-peptide was purified on HPLC using acetonitrile/water (0.1% TFA) gradient (20-50% acetonitrile in 30 minutes). Purified Biotin-peptide amide in 5 mL DMF (27.5 mg, 20 μmol) was coupled with 10 mg (20 μmol) DTAF in the presence of 17.5 μL (100 μmol) DIPEA for 12 hour. The reaction mixture was filtered and diluted with 5 mL 50% acetic acid and 20 mL water. This solution was purified on HPLC using acetonitrile/water (0.1% TFA) gradient (20-50% acetonitrile in 30 minutes). The identity of Biotin-γ-Abu-Ser-Gln-Asn-Tyr-Pro-Ile-Val-Gln-Lys(DTAF)-NH₂ [SEQ ID NO:4] was confirmed by FAB mass spectrometry: M+H=1844.6.

Recombinant HCMV protease was purified from E. coli expressing a DNA construction encoding the protease domain of the U_{L}80 open reading frame of human cytomegalovirus strain AD169. The construction also encoded six additional histidine residues at the amino terminus of the protease. These additional histidine residues provided an affinity ligand by which it was purified using nickel-nitriloacetic acid-agarose (Qiagen).

### Assemblin Screening Assay Protocol

The assay was performed in a buffer consisting 10 mM Phosphate buffer, pH 7.4, 20% glycerol, 150 mM sodium acetate, 0.1% CHAPS, 0.1 mM EDTA, 0.05% BSA, 2 mM NaSO₃. The pH of the buffer was adjusted to 7.4 before the addition of glycerol.

The purified protease was stored as a 10 μM stock solution in 50/50 (V/V) glycerol/water, 50 μL per vial, and held at −20° C. A positive displacement pipette was used to make the 50 μL aliquots. This stock was diluted with assay buffer to 32 nM. A 100 μL aliquot of this solution was used in the enzyme reaction. A 150 μM substrate stock solution was prepared in assay buffer and stored at 4° C. in the dark. A 20 μM dilute assay stock was prepared by dilution of the 150 μM storage stock. A 5 mg/mL avidin stock solution was made by dissolving avidin (Molecular Probes) in assay buffer and stored in the refrigerator at 4° C. Roundbottom 96-well plate microtiter plates (Black MicroFluor, Dynatec) were pre-blocked with 1.0% BSA in PBS, pH 7.4, and stored at 4° C. The plates were rinsed and dried before use.

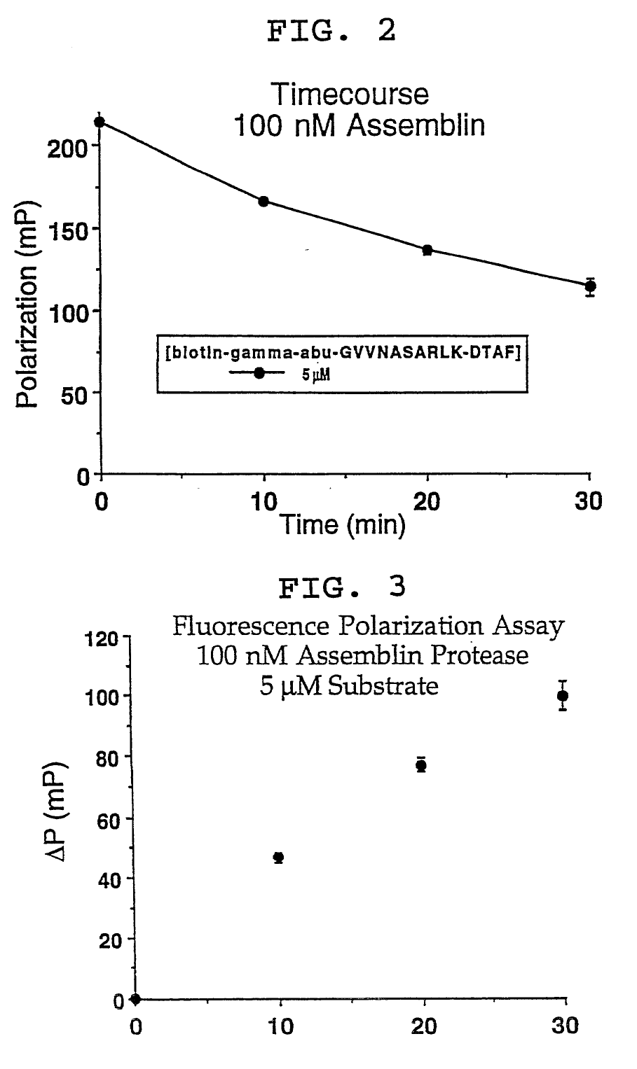

An HCMV protease (also known as assemblin) encoded by U_{L}80, in buffer (100 nM) was added in multiple wells to rinsed and dried, pre-blocked microtiter plate. No precautions are taken to stabilize the temperature. Substrate (5 μM final concentration) was added, mixed 5 times, and incubated at room temperature. Avidin (35 μL of 5 mg/mL) was added to one group of wells after 10 minutes, to a second group of wells after 20 minutes and to the remaining wells after thirty minutes, mixed 5 times, and the polarization of the resulting mixture was measured on the FPM2 fluorescence polarimeter. The results are shown in Table 1 and FIGS. 2-3. The complete hydrolysis to Ser-Ala-Leu-Arg-Lys(DTAF)-NH₂ gave a Pₘᵢₙ value of 32.1 mP.

TABLE 1

| Time | Polarization | ΔP = Pₘₐₓ − P(t) |
|---|---|---|
| (min.) | (mP) | (mP) |
| 0 | 214.1 | 0.0 |
| 10 | 167.2 | 47.0 |
| 20 | 136.9 | 77.2 |
| 30 | 114.2 | 99.9 |

All mentioned references are incorporated by reference as if here written.

Although this invention has been described with respect to specific embodiments, the details of these embodiments are not to be construed as limitations.

What is claimed is:

1. A method for determining the activity of a protease, said method comprising
a) incubating a mixture of said protease and a substrate capable of being bound to an anchor, said substrate having a fluorescent radical attached thereto;
b) binding the substrate to an anchor;
c) measure the fluorescence polarization of the mixture.

2. The method of claim 1 wherein the substrate is selected from compounds of Formula I
Z—(W)ₘ—X—(V)ₙ—Y (I)
wherein X is an amino acid sequence sufficient for substrate recognition by a protease; wherein V and W are independently selected from aminoalkylcarboxylic acids; wherein m and n are numbers independently selected from 0 and 1; and wherein one of Y and Z is a fluorescent radical and the other is a binding radical.

3. The method of claim 2 wherein X is a peptide containing six to sixteen amino acids, inclusive; and wherein V and W are independently selected from glycine, 4-aminobutyric acid, 5-aminopentanoic acid, 6-aminocaproic acid and 7-aminoheptanoic acid.

4. The method of claim 3 wherein the anchor is selected from a biotin selective protein, a solid support, and an antibody; wherein the binding radical is selected from biotin, digoxigenin and radicals capable of binding to a solid support; and wherein the fluorescent radical is selected from derivatives of fluorescein, rhodamine, coumarin, eosin, pyrene, quinoline, DANSYL, dinitrophenyl, benzimidazole, DABCYL, EDANS, cascade blue, Texas red, acidine orange and BODIPY.

5. The method of claim 4 wherein the fluorescent radical is a fluorescein derivative.

6. The method of claim 5 wherein the biotin selective protein is avidin or streptavidin; wherein the binding radical is biotin; and wherein the fluorescent radical is DTAF.

7. The method of claim 1 wherein the proteases are viral proteases.

8. The method of claim 7 wherein the proteases are selected from HIV proteases and herpes proteases.

9. The method of claim 8 wherein the herpes viruses proteases are selected from HCMV proteases, MCMV proteases, HSV-1 proteases and HSV-2 proteases.

10. The method of claim 6 wherein the substrates are selected from biotin-γ-Abu-Gly-Val-Val-Asn-Ala-Arg-Ser-Leu-Lys(DTAF)-NH₂ [SEQ ID NO:3] and biotin-γ-Abu-Ser-Gln-Asn-Tyr-Pro-Ile-Val-Gln-Lys(DTAF)-NH₂ [SEQ ID NO:4].

11. A method for identifying compounds which inhibit a protease, said method comprising a) incubating a mixture of said protease, the compound, and a substrate having both a fluorescent radical and a radical capable of binding to an anchor; b) binding the substrate to the anchor; c) measure the fluorescence polarization of emitted light; and d) calculating the amount of protease inhibition.

12. A compound of Formula I
Z—(W)ₘ—X—(V)ₙ—Y (I)
wherein X is an amino acid sequence sufficient for substrate recognition by a protease; wherein V and W are independently selected from aminoalkylcarboxylic acids; wherein m and n are numbers independently selected from 0 and 1; and wherein one of Y and Z is a fluorescent radical and the other is a binding radical.

13. The compound of claim 12 wherein X is a peptide containing six to sixteen amino acids, inclusive; wherein V and W are independently selected from glycine, 4-aminobutyric acid, 5-aminopentanoic acid, 6-aminocaproic acid and 7-aminoheptanoic acid; wherein the binding radical is biotin; and wherein the fluorescent radical is a fluorescein derivative.

14. The compound of claim 13 which is biotin-γ-Abu-Gly-Val-Val-Asn-Ala-Arg-Ser-Leu-Lys(DTAF)-NH₂ [SEQ ID NO:3].

15. The compound of claim 13 which is biotin-γ-Abu-Ser-Gln-Asn-Tyr-Pro-Ile-Val-Gln-Lys(DTAF)-NH₂ [SEQ ID NO:4].
